(19) United States
(12) Patent Application Publication
(10) Pub. No.: US 2010/0099193 A1
(43) Pub. Date: Apr. 22, 2010

(54) SYSTEM AND METHOD FOR MONITORING AND CONTROLLING QUALITY OF CULTURE WATER AND INTEGRATED WATER QUALITY ANALYZER THEREOF

(75) Inventors: Kun-Cheng Hsu, Pingtung County (TW); Yung-Ching Huang, Taipei City (TW); Yu-Hsien Chiu, Kaohsiung County (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: INDUSTRIAL TECHNOLOGY RESEARCH INSTITUTE, Hsinchu (TW)

(21) Appl. No.: 12/342,003

(22) Filed: Dec. 22, 2008

(30) Foreign Application Priority Data
Oct. 20, 2008 (TW) 97140165

(51) Int. Cl. G01N 35/02; G01N 33/18; G21C 17/00; G01N 21/00

(52) U.S. Cl. 436/47; 422/62; 422/82.05; 422/64; 702/182

(57) ABSTRACT

A system and a method for monitoring and controlling the quality of a culture water are provided. The system includes a plurality of smart culture nodes having integrated water quality analyzers, a culture gateway and a terminal host. In the present method, each of the smart culture nodes detects the quality of the culture water through the integrated water quality analyzer thereof and activates corresponding culture equipments to adjust the quality of the culture water. Besides, the terminal host groups the smart culture nodes through the culture gateway and synchronously controls the smart culture nodes in the same group according to a water quality information collected by the smart culture nodes in the same group so as to activate the corresponding culture equipments to adjust the quality of the culture water. Thereby, the quality of the culture water can be efficiently monitored and controlled.

### CROSS-REFERENCE TO RELATED APPLICATION

This application claims the priority benefit of Taiwan application serial No. 97140165, filed on Oct. 20, 2008. The entirety of the above-mentioned patent application is hereby incorporated by reference herein and made a part of this specification.

### BACKGROUND OF THE INVENTION

1. Field of the Invention

The present invention generally relates to a system and a method for monitoring and controlling the quality of a culture water by grouping and synchronously monitoring and controlling smart culture nodes and an integrated water quality analyzer and a water quality analysis method thereof.

2. Description of Related Art

In today's aquaculture industry, many tasks, such as feeding, changing water, spreading fertilizer, and supplying air, are carried out manually. In order to improve the efficiency of aquaculture, many electronic sensors and water quality reagents are adopted to collect different water quality information of culture ponds or fish tanks, and many different automatic devices (for example, actuators) are adopted to allow different culture equipments, such as filters, feeders, air pumps, and air supply valves, to operate automatically. However, existing aquatic electronic equipments have very low expandability and have to be set manually. Accordingly, along with the increase in the scale of culture units, setting these electronic sensors and actuators will become very complicated and time-consuming due to the close relationship between culture water in different areas of the culture unit.

For example, fishes, prawns, and float grass may coexist in a culture unit (for example, a large culture pond or a fish tank). Since these creatures may come from different regions and accordingly require different water conditions, the ecological composition of the culture unit may be very complicated. If a novice takes care of such a culture unit, the creatures in the culture unit may die of inappropriate settings of the water conditions. Even an experienced person may make mistakes in the settings of the water conditions with such a complicated ecological composition and the creatures in the culture unit may die of such mistakes.

Conventionally, the setting of environmental parameters, the examination of growth state, the delivery of drugs, and the application of fertilizer after the culture process starts all rely on human judgement. However, it is difficult to pass down human experience, systematically record the culture process of each culture unit, and quantify various culture parameters.

In order to resolve foregoing problems, a system and a method for automatically detecting water quality anomaly, analyzing water quality, and providing feedback control and for monitoring an aquatic environment through cable or wireless transmission are respectively disclosed in U.S. Pat. No. 7,222,047 and No. 2005/0172910. In addition, a system and a method for detecting water quality anomaly and automatically reporting aquaculture information through wireless transmission are disclosed in Taiwan patent No. 305111. Moreover, a system and a method for remotely monitoring and controlling a fishery through a Global System for Mobile Communications (GSM) module is disclosed in Taiwan patent No. 200614911, and an aquaculture management method by adopting a water quality analysis and image-assisted monitoring and controlling mechanism through the Internet is disclosed in Taiwan patent No. 095102893. However, even all of these conventional techniques allow a culturist to remotely and conveniently monitor and control the quality of a culture water, they do not provide an efficient monitoring and controlling mechanism regarding a large culture pond or multiple fish tanks or an efficient method for culturists to pass down or exchange aquaculture experiences.

### SUMMARY OF THE INVENTION

Consistent with the invention, there is provided a system and a method for monitoring and controlling the quality of a culture water, wherein a water quality information of the culture water can be efficiently monitored and controlled in a group manner.

Consistent with the invention, there is provided a system for monitoring and controlling the quality of a culture water. The system includes a plurality of smart culture nodes, a culture gateway, and a terminal host. Each of the smart culture nodes is connected to a culture equipment and includes a water quality parameter regulator, an integrated water quality analyzer, and an actuator. The water quality parameter regulator sets an environmental parameter. The integrated water quality analyzer is electrically connected to the water quality parameter regulator for detecting a water quality information of the culture water. The actuator is electrically connected to the water quality parameter regulator for starting the culture equipment. The terminal host issues a control instruction to set the environmental parameters of the smart culture nodes. The culture gateway is connected between the smart culture nodes and the terminal host for relaying the water quality information and the control instruction. The terminal host controls the culture gateway to group at least part of the smart culture nodes into a culture group and synchronously controls the smart culture nodes in the culture group according to the water quality information collected by the smart culture nodes in the culture group to start the corresponding culture equipments.

Also consistent with the invention, there is provided a method for monitoring and controlling the quality of a culture water. The method is suitable for foregoing system. The method includes: remotely setting the environmental parameters of the smart culture nodes through the culture gateway by using the terminal host; detecting the water quality information by using each of the smart culture nodes; and controlling the culture gateway by using the terminal host to group at least a part of the smart culture nodes into a culture group and synchronously controlling the smart culture nodes in the culture group according to the water quality information collected by the smart culture nodes in the culture group to start the corresponding culture equipments.

Further, and consistent with the invention, there is provided a system for monitoring and controlling the quality of a culture water. The system is suitable for monitoring and controlling a water quality information of the culture water. The system includes a plurality of actuators, a water quality parameter regulator, an integrated water quality analyzer, a terminal host, and a culture gateway. The actuators are respectively connected to a plurality of culture equipments. The water quality parameter regulator is electrically connected to the actuators and has a plurality of environmental parameters corresponding to the actuators. The integrated water quality analyzer is electrically connected to the water quality parameter regulator for detecting a water quality information of the culture water. The terminal host issues a control instruction to set the environmental parameters. The culture gateway is connected between the water quality parameter regulator and the terminal host for relaying the control instruction. The water quality parameter regulator starts the corresponding culture equipments through the actuators according to the water quality information and the environmental parameters.

Also consistent with the invention, there is provided a method for monitoring and controlling the quality of a culture water. The method is suitable for foregoing system. The method includes: setting the environmental parameters of the water quality parameter regulator; obtaining the water quality information through the integrated water quality analyzer; comparing the water quality information with the environmental parameters; and determining whether to activate the actuators and operation time of the actuators according to the comparison result to start the corresponding culture equipments.

Further, and consistent with the invention, there is provided an integrated water quality analyzer including a water quality analysis controller, a water extraction structure, a test water guiding structure, a reagent guiding structure, and an optical analysis device. The water extraction structure is electrically connected to the water quality analysis controller for extracting a test water through a water extraction pipeline. The test water guiding structure is electrically connected to the water quality analysis controller, and the test water guiding structure obtains the test water from the water extraction structure through a water pipeline and injects the test water into at least one test container. The reagent guiding structure is electrically connected to the water quality analysis controller and has at least one water quality reagent, and the reagent guiding structure injects the water quality reagent into the test containers. The optical analysis device is electrically connected to the water quality analysis controller for analyzing the test water in the test containers to obtain a water quality information.

Also consistent with the invention, there is provided a water quality analysis method, suitable for foregoing integrated water quality analyzer. The water quality analysis method includes: controlling the water extraction structure by using the water quality analysis controller to obtain the test water through the water extraction pipeline; evenly injecting the test water into the test containers by using the water extraction structure; automatically injecting different water quality reagents into the corresponding test containers by using the reagent guiding structure; and analyzing the test water in the test containers by using the optical analysis device to generate the water quality information of the test water.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a diagram of a system for monitoring and controlling the quality of a culture water according to a first embodiment of the present invention.

FIG. 2 is a schematic block diagram of a smart culture node according to the first embodiment of the present invention.

FIG. 3 is a block diagram of an automatic optical water quality analyzer according to the first embodiment of the present invention.

FIG. 4 is a flowchart illustrating a process for analyzing the quality of a culture water by using an automatic optical water quality analyzer according to the first embodiment of the present invention.

FIG. 5 is a flowchart of a method for monitoring and controlling the quality of a culture water according to the first embodiment of the present invention.

FIG. 6 illustrates the steps for setting environmental parameters according to an embodiment of the present invention.

FIG. 7 is a diagram of a system for monitoring and controlling the quality of a culture water according to a second embodiment of the present invention.

FIG. 8 is a flowchart illustrating a process for setting a culture group according to the second embodiment of the present invention.

FIG. 9A illustrates a method for monitoring and controlling the quality of a culture water, wherein smart culture nodes are grouped through a first grouping method.

FIG. 9B illustrates a method for monitoring and controlling the quality of a culture water, wherein smart culture nodes are grouped through a second grouping method.

### DESCRIPTION OF THE EMBODIMENTS

Exemplary embodiments consistent with the present invention provides a system and a method for monitoring and controlling the quality of a culture water, wherein a water quality information of the culture water is efficiently monitored and controlled, and a mechanism for passing down and exchanging culturists' experiences is provided.

In exemplary embodiments consistent with the present invention provides an integrated water quality analyzer and a method thereof, wherein a water quality information of a test water is automatically analyzed by using water quality reagents and an optical analysis device.

In exemplary embodiments consistent with the present invention, smart culture nodes are grouped through a culture gateway so as to remotely and synchronously control those smart culture nodes belonging to the same culture unit or to synchronously monitor and control specific water quality items of the smart culture nodes in an culture environment. As a result, the efficiency in monitoring and controlling water quality is improved.

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments consistent with the present invention do not represent all implementations consistent with the invention. Instead, they are merely examples of systems and methods consistent with aspects related to the invention as recited in the appended claims.

### First Embodiment

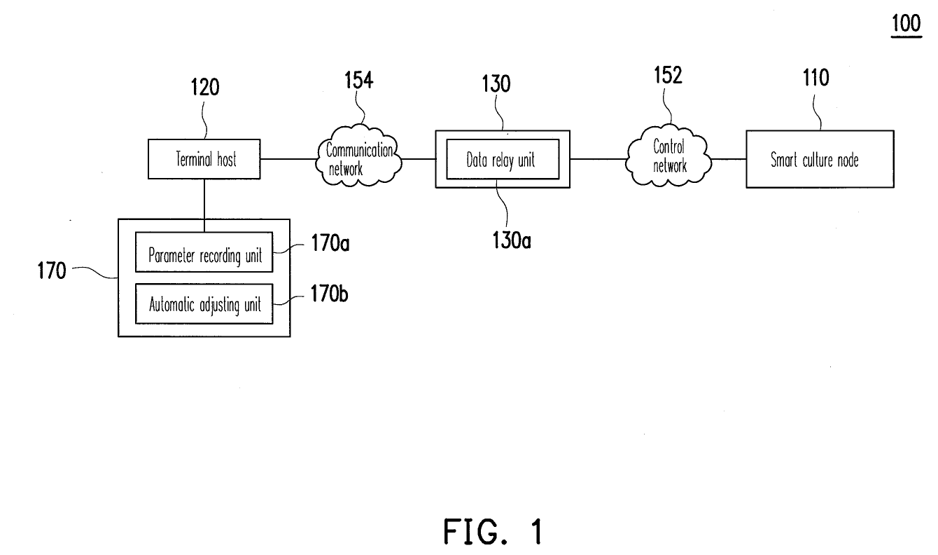

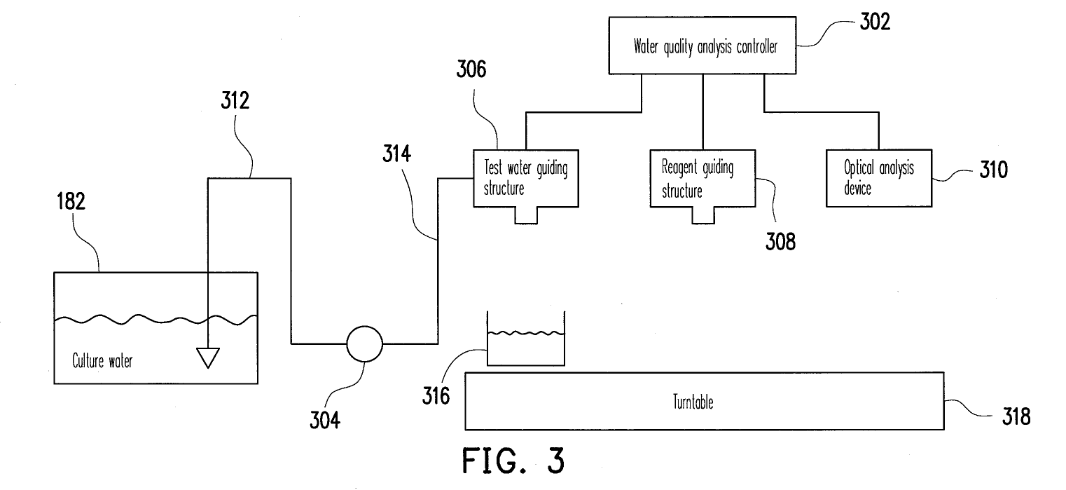

FIG. 1 is a diagram of a system for monitoring and controlling the quality of a culture water according to the first embodiment consistent with the present invention. In the present embodiment, the smart culture node 110 of the water quality monitoring and controlling system 100 is installed in a small fish tank 182 (as shown in FIG. 3). The water quality monitoring and controlling system 100 detects a water quality information of the culture water in the fish tank 182 and performs corresponding control actions to the fish tank 182 according to the water quality information.

Referring to FIG. 1, the water quality monitoring and controlling system 100 includes the smart culture node 110, a terminal host 120, and a culture gateway 130.

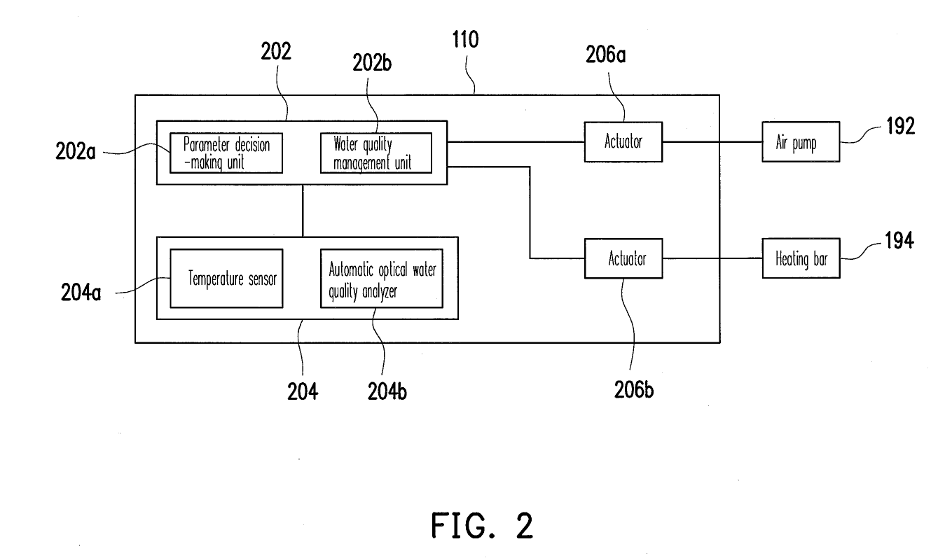

The smart culture node 110 detects the water quality information of the culture water and connects to a culture equipment according to the setting and control instructed by the terminal host 120. The culture equipment is an equipment used for adjusting the water quality (for example, the water volume, the dissolved oxygen content, the carbon dioxide concentration, the pH value, and the temperature of a culture unit) of a culture water. The culture equipment may be a lamp, an electromagnetic valve for controlling carbon dioxide, a heating bar, an automatic feeder, an air pump, or a water cooler. Namely, the smart culture node 110 may be connected to one or multiple of foregoing culture equipments for detecting and adjusting the water quality. In the present embodiment, an air pump 192 and a heating bar 194 (as shown in FIG. 2) are connected to the smart culture node 110 for regulating the dissolved oxygen content and the temperature of the culture water in the fish tank 182 under the control of the smart culture node 110. It should be mentioned that in the present embodiment, even though the water quality monitoring and controlling system 100 includes only one smart culture node, it may also include multiple smart culture nodes and individually control the operation of each of the smart culture nodes.

FIG. 2 is a schematic block diagram of a smart culture node according to the first embodiment of the present invention.

Referring to FIG. 2, the smart culture node 110 includes a water quality parameter regulator 202, an integrated water quality analyzer 204, and actuators 206a and 206b.

The water quality parameter regulator 202 sets environmental parameters of culture equipments connected to the smart culture node 110. The water quality parameter regulator 202 includes a parameter decision-making unit 202a and a water quality management unit 202b coupled to each other. The parameter decision-making unit 202a categorizes the water quality information detected by the integrated water quality analyzer 204, and the water quality management unit 202b controls the corresponding actuators 206a and 206b.

To be specific, in the present embodiment, the water quality parameter regulator 202 receives a control instruction of the terminal host 120 through the culture gateway 130 to set the environmental parameters of the corresponding culture equipments (i.e., the air pump 192 and the heating bar 194), and the water quality parameter regulator 202 determines whether to activate the actuators 206a and 206b, so as to start or stop the culture equipments and adjust the water quality of the fish tank 182, according to the current water quality information detected by the integrated water quality analyzer 204 and the preset environmental parameters. For example, in the present embodiment, a culturist sets an expected dissolved oxygen content value and an expected temperature as the environmental parameters through an operation interface of the terminal host 120.

The integrated water quality analyzer 204 is electrically connected to the water quality parameter regulator 202. The integrated water quality analyzer 204 obtains the water quality information of the culture water. In the present embodiment, the water quality information to be detected is water temperature and dissolved oxygen content. Accordingly, in the present embodiment, the integrated water quality analyzer 204 includes a temperature sensor 204a and an automatic optical water quality analyzer 204b. The temperature sensor 204a is an electronic sensor which can detect the temperature of a culture water, and the automatic optical water quality analyzer 204b is used for detecting the dissolved oxygen content in the culture water. However, the present invention is not limited thereto, and other sensors may also be adopted in the present embodiment for obtaining other water quality information, such as a pH value.

It should be mentioned that regarding water quality analysis, some water quality information (for example, the pH value and temperature) can be detected by using electronic detectors (for example, sensors), while some other water quality information (for example, the oxygen content and nitrogen compound content) has to be manually detected by using water quality reagents. Thus, the conventional water quality analysis process is very inconvenient and error-prone. In the present embodiment, the automatic optical water quality analyzer 204b is a device which can automatically detect water quality by using water quality reagents.

FIG. 3 is a block diagram of an automatic optical water quality analyzer according to the first embodiment of the present invention.

Referring to FIG. 3, the automatic optical water quality analyzer 204b includes a water quality analysis controller 302, a water extraction structure 304, a test water guiding structure 306, a reagent guiding structure 308, and an optical analysis device 310.

The water quality analysis controller 302 automatically controls the water extraction structure 304, the test water guiding structure 306, the reagent guiding structure 308, and the optical analysis device 310 under the control of the terminal host 120 to analyze the quality of a test water.

The water extraction structure 304 is electrically connected to the water quality analysis controller 302, and the water extraction structure 304 extracts the culture water from a culture environment (i.e., the fish tank 182) through a water extraction pipeline 312 and sends the culture water to the test water guiding structure 306 through a water pipeline 314 under the control of the water quality analysis controller 302.

The test water guiding structure 306 is electrically connected to the water quality analysis controller 302, and the test water guiding structure 306 guides the culture water received from the water extraction structure 304 into a test container 316. In the present embodiment, only one test container 316 is used since the automatic optical water quality analyzer 204b only detects the oxygen content in the culture water. However, the present invention is not limited thereto, and in another embodiment of the present invention, if multiple water quality items are to be detected, multiple test containers are then disposed in the automatic optical water quality analyzer 204b, and the test water guiding structure 306 distributes the culture water evenly into these test containers.

The reagent guiding structure 308 is electrically connected to the water quality analysis controller 302 and has a water quality reagent for detecting the dissolved oxygen content. The reagent guiding structure 308 injects the water quality reagent into the test container 316 under the control of the water quality analysis controller 302. Similarly, in another embodiment of the present invention, if multiple water quality items are to be detected, different water quality reagents (for example, a nitrogen compound reagent and a dissolved oxygen reagent) are then used by the reagent guiding structure 308.

The optical analysis device 310 is electrically connected to the water quality analysis controller 302 for analyzing the culture water in the test container 316 to obtain the water quality information. To be specific, a charge-coupled device (CCD) is disposed in the optical analysis device 310 for detecting the color image change of the culture water injected with the water quality reagent and analyzing the water quality information according to the characteristics of the water quality reagent. It should be mentioned that in an embodiment of the present invention, the device for detecting the color image change may also be implemented as a complementary metal-oxide semiconductor (CMOS) image sensor.

In an embodiment of the present invention, the automatic optical water quality analyzer 204b further includes a turntable 318 for placing the test container 316 so as to automatically move the test container 316 and carry out the injection of the water quality reagent and the optical analysis.

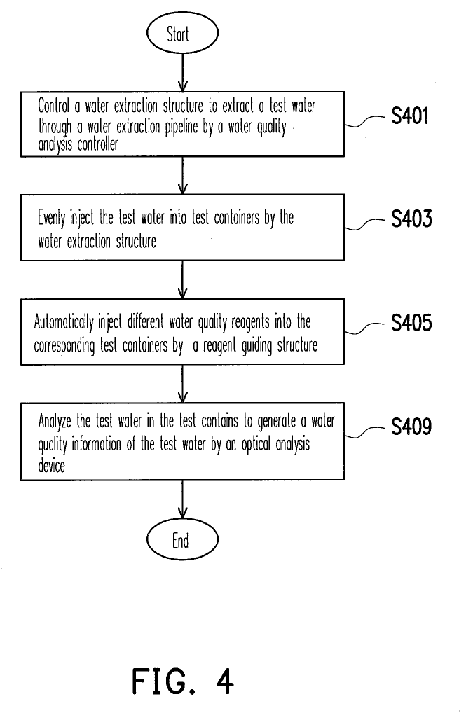

FIG. 4 is a flowchart illustrating a process for analyzing a water quality by using an automatic optical water quality analyzer according to the first embodiment of the present invention.

Referring to FIG. 4, in step 401, the water quality analysis controller 302 controls the water extraction structure 304 to obtain a test water through the water extraction pipeline 312. Then, in step S403, the water extraction structure 304 distributes the test water evenly into the test containers. Next, in step S405, the reagent guiding structure 308 automatically injects different water quality reagents into the corresponding test containers. Finally, in step S407, the optical analysis device 310 analyzes the test water in the test containers to generate a water quality information of the test water.

Referring to FIG. 2 again, the actuators 206a and 206b are electrically connected to the water quality parameter regulator 202 for starting the culture equipments connected thereto. In the present embodiment, the air pump 192 and the heating bar 194 are respectively connected to the actuators 206a and 206b. To be specific, the water quality parameter regulator 202 can start and stop the operations of the air pump 192 and the heating bar 194 through the actuators 206a and 206b.

Referring to FIG. 1 again, the terminal host 120 issues a control instruction to the smart culture node 110 to set aforementioned environmental parameters. In the present embodiment, the terminal host 120 provides a user operation software (not shown) such that the culturist can connect to the smart culture node 110 and set the expected dissolved oxygen content and water temperature through the user operation software. In addition, in an embodiment of the present invention, the water quality monitoring and controlling system 100 further includes an environment learning module 170. The environment learning module 170 includes a parameter recording unit 170a for recording the regulation process of the environmental parameters and an automatic adjusting unit 170b for loading a setting file stored in the parameter recording unit 170a to set the environmental parameters. In particular, the parameter recording unit 170a may store data regarding the culture environment suitable for different aquatic creatures.

In the present embodiment, all the equipments in the smart culture node 110 are connected through a control network 152 (e.g. a control area network). Thus, in order to allow the terminal host 120 to remotely control the smart culture node 110, in the present embodiment, the culture gateway 130 is adopted for connecting the smart culture node 110 and the terminal host 120. In an embodiment of the present invention, the culture gateway 130 includes a data relay unit 130a for relaying the control instruction from the terminal host 120 to the smart culture node 110 or the water quality information from the smart culture node 110 to the terminal host 120.

To be specific, the terminal host 120 is connected to the culture gateway 130 through a communication network 154. Thus, in the present embodiment, the terminal host 120 remotely controls the smart culture node 110 connected through the control network 152 through the relay of the communication network and the culture gateway 130. In an embodiment of the present invention, the communication network 154 is a communication network using Internet Protocol (IP). However, a non-IP communication network may also be applied to the present invention. In addition, the culturist may further use a mobile device (for example, a personal digital assistant (PDA)) to obtain information of the culture environment through the communication network 154 if the communication network 154 adopts a wireless network standard.

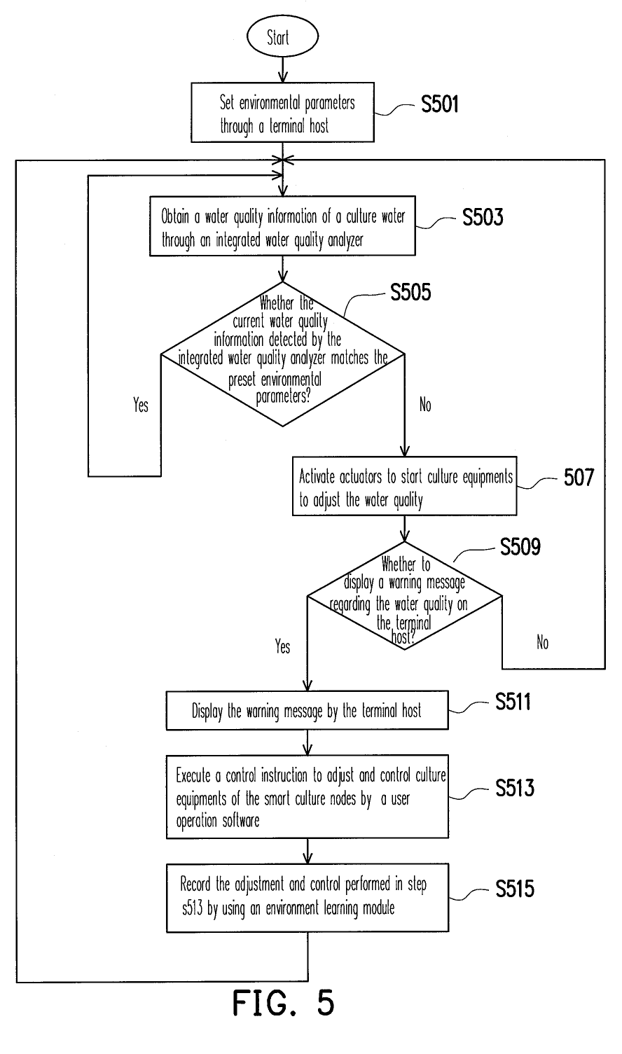

FIG. 5 is a flowchart of a method for monitoring and controlling the quality of a culture water according to the first embodiment of the present invention.

Referring to FIG. 5, in step S501, the environmental parameters of the water quality parameter regulator 202 are set to the smart culture node 110 on the user operation software of the terminal host 120. To be specific, after installing foregoing hardware equipments (as shown in FIG. 1), the culturist has to set the environmental parameters according to the environment in the fish tank 182 and the characteristics of the aquatic creatures to be cultured. In the example of storing the environmental parameters in the environment learning module 170, the culturist may directly load the setting file provided by another culturist, or the culturist may also set the environmental parameters through following steps.

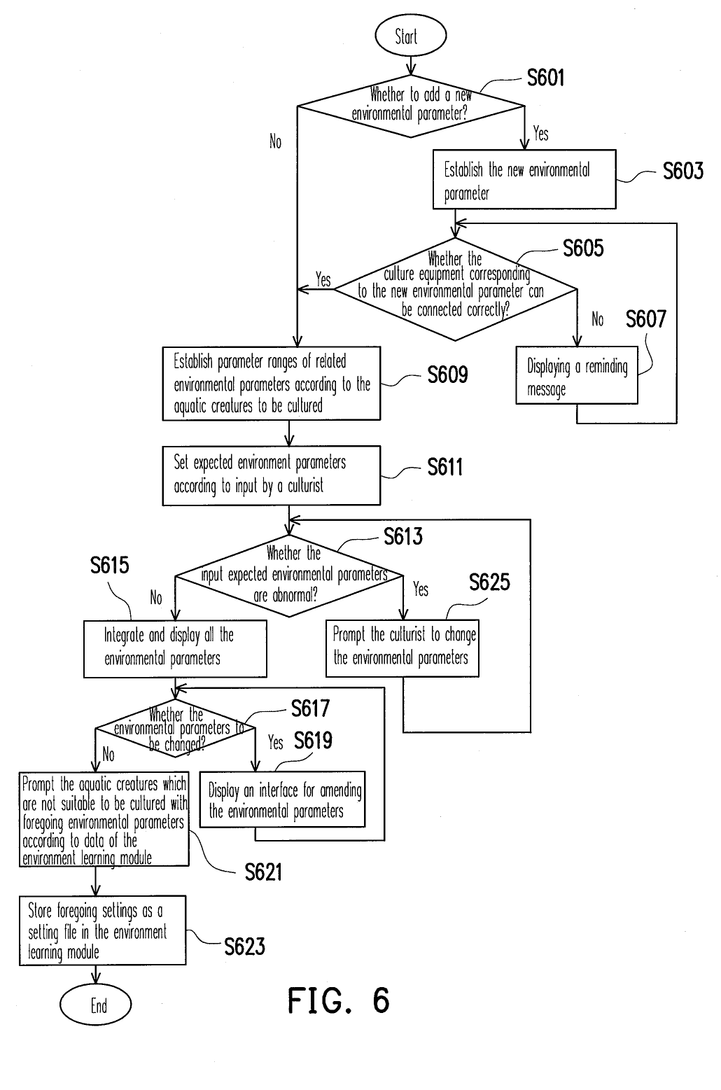

FIG. 6 illustrates the steps for setting the environmental parameters according to an embodiment of the present invention. Referring to FIG. 6, in step S601, the user operation software determines whether to add a new environmental parameter.

If the user operation software determines in step S601 that the culturist is about to add a new environmental parameter, in step S603, the user operation software establishes the new environmental parameter, and in step S605, the user operation software determines whether the culture equipment corresponding to the new environmental parameter can be correctly connected.

If it is determined in step S605 that the culture equipment corresponding to the new environmental parameter cannot be correctly connected, in step S607, the user operation software displays a reminding message to let the culturist to confirm and then returns to step S605.

Next, in step S609, parameter ranges of related environmental parameters are established according to the creatures to be cultured. In the present embodiment, settings of water temperature and dissolved oxygen content are described as an example. However, as described above, the environmental parameters may further include the size or volume, the carbon dioxide concentration, the pH value, the illumination pattern, the feed quantity, the temperature range, and a timing device of a culture unit.

After that, in step S611, the user operation software sets the expected environmental parameters according to the input of the culturist, and in step 613, the user operation software determines whether the input expected environmental parameters are abnormal according to the parameter ranges established in step S609.

If the user operation software determines in step S613 that the input environmental parameters are abnormal, in step S625, the user operation software prompts the culturist to change the input and then returns to step S613.

If the user operation software determines in step S613 that the environmental parameters are normal, then in step S615, the user operation software integrates and displays all the environmental parameters. In step S617, the user operation software requests the culturist to confirm.

If the user operation software determines in step S617 that the culturist is about to change the environmental parameters, in step S619, the user operation software displays an interface for amending the environmental parameters and then returns to step S617.

If the user operation software determines in step S617 that the culturist has finished the setting of the environmental parameters, in step S621, the user operation software displays the creatures which are not suitable to be cultured with foregoing environmental parameters according to the data in the environment learning module 170. Finally, in step S623, the user operation software stores foregoing settings as a setting file into the environment learning module 170. In particular, the setting file can be used for exchanging culture experiences with other culturists. In addition, when subsequently the culturist adds a new culture equipment, the environmental parameter corresponding to the new culture equipment can be added through the steps illustrated in FIG. 6.

Referring to FIG. 5 again, in step 503, a water quality information of the culture water is obtained through the integrated water quality analyzer 204. For example, in the present embodiment, the temperature of the culture water in the fish tank 182 is detected through the temperature sensor 204a of the integrated water quality analyzer 204, and the dissolved oxygen content of the culture water in the fish tank 182 is detected through the automatic optical water quality analyzer 204b of the integrated water quality analyzer 204. After that, in step S505, the water quality parameter regulator 202 compares the current water quality information detected by the integrated water quality analyzer 204 with the preset environmental parameters. If the water quality parameter regulator 202 determines in step S505 that the current water quality information does not match the preset environmental parameters, in step S507, the water quality parameter regulator 202 activates the actuator 206a or 206b to start the air pump 192 or the heating bar 194 so as to adjust the water quality. If the water quality parameter regulator 202 determines in step S505 that the current water quality information matches the environmental parameters, the water quality parameter regulator 202 takes no action and step S503 is executed again after certain time to carry on the monitoring and controlling task.

In step S501, besides setting the environmental parameters, the culturist may further sets a prompt setting of the water quality information. For example, when the temperature exceeds an expected value, the turbidity exceeds an expected value, the concentration of a compound exceeds an expected value, or the content of certain element is too low, the smart culture node 110 sends the information to the terminal host 120 to prompt the culturist. Thus, in step S509, whether to display a warning message regarding the water quality on the terminal host 120 is determined according to the water quality information.

If it is determined in step S509 that the culturist is not to be prompted, step S503 is executed. If it is determined in step S509 that the culturist is to be prompted, in step S511, the terminal host 120 displays the warning message, and in step S513, the culturist executes a control instruction through the user operation software to adjust and control various culture equipments of the smart culture node 110.

Next, in step S515, the environment learning module 170 records the adjustment and control carried out in step S513 and then executes step S503.

As described above, in the present embodiment, after setting the smart culture node 110 through the terminal host 120, the smart culture node 110 can monitor and control the quality of the culture water in the fish tank 182. In addition, the culturist may also fine-tune the quality of the culture water in the fish tank 182 manually, and meanwhile, the environment learning module 170 may record the fine-tuned regulation process and subsequently set the smart culture node 110 by using the recorded regulation process when similar condition is encountered.

### Second Embodiment

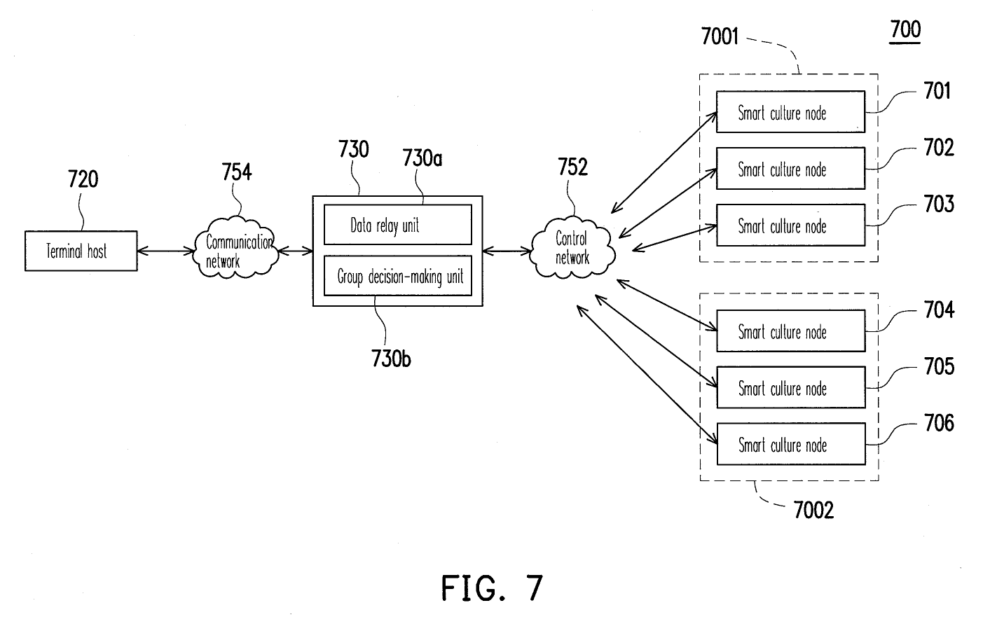

FIG. 7 is a diagram of a system for monitoring and controlling the quality of a culture water according to the second embodiment of the present invention. In the present embodiment, the smart culture nodes 701˜706 of the water quality monitoring and controlling system 700 are installed in a culture environment having a large culture pond and a plurality of fish tanks (i.e., a first fish tank, a second fish tank, and a third fish tank). The water quality monitoring and controlling system 700 detects a water quality information of the culture pond and the fish tanks and appropriately executes corresponding control actions to the large culture pond and the first, the second, and the third fish tank according to the detected water quality information.

Referring to FIG. 7, the water quality monitoring and controlling system 700 includes the smart culture nodes 701˜706, a terminal host 720, and a culture gateway 730.

The smart culture nodes 701˜706, the terminal host 720, and the culture gateway 730 respectively have all the functions of the smart culture node 110, the terminal host 120, and the culture gateway 130 in the first embodiment and these functions thereof will not be described herein. Below, only the differences between these like elements in the present embodiment and the first embodiment are described.

In the present embodiment, the user operation interface of the terminal host 720 offers a function for grouping the smart culture nodes 701˜706. Namely, the terminal host 720 can synchronously set and control the smart culture nodes in unit of groups.

In order to achieve foregoing grouping function, in an embodiment of the present invention, the culture gateway 730 further includes a group decision-making unit 730b besides a data relay unit 730a. The group decision-making unit 730b groups the smart culture nodes connected thereto according to a control instruction received from the terminal host 720. To be specific, the terminal host 720 controls the culture gateway 730 to group the smart culture nodes into different culture groups and synchronously controls the smart culture nodes in the same culture group according to the water quality information detected by the smart culture nodes in the same culture group so as to start the corresponding culture equipments.

For example, in the present embodiment, the smart culture nodes 701˜703 are used for monitoring and controlling the large culture pond. In the present embodiment, since the culture water in the culture pond flows around, the smart culture nodes 701˜703 are set as a first culture group 7001, and the terminal host 720 can synchronously control the culture equipments of all the smart culture nodes in the first culture group 7001.

In addition, the smart culture nodes 704˜706 may be respectively disposed in the first fish tank, the second fish tank, and the third fish tank; but for certain reasons, a specific environmental parameter of the first fish tank, the second fish tank, and the third fish tank has to be regulated synchronously (for example, the water temperature of the first fish tank, the second fish tank, and the third fish tank has to be increased together when a cold current comes). Thus, the smart culture nodes 704˜706 are set as a second culture group 7002, and the terminal host 720 can synchronously control all the smart culture nodes in the second culture group 7002.

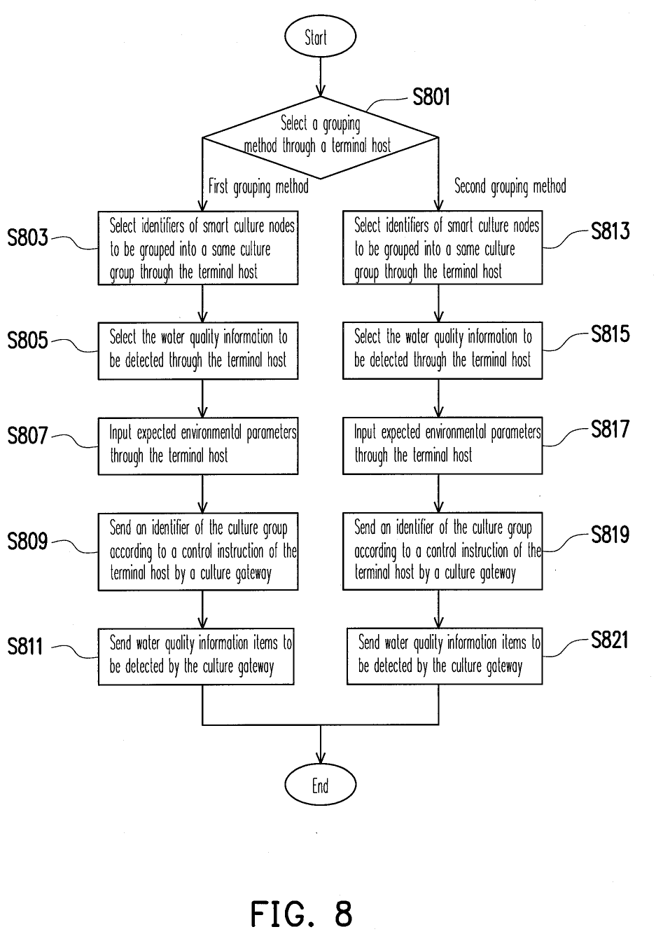

FIG. 8 is a flowchart illustrating how to set a culture group according to the second embodiment of the present invention.

Referring to FIG. 8, in step S801, the culturist selects the grouping method to be executed on the user operation software of the terminal host 720. In the present embodiment, the methods for grouping the smart culture nodes include a first grouping method (i.e., to group a plurality of smart culture nodes in a single culture pond) and a second grouping method (i.e., to group a specific environmental parameter belonging to a plurality of smart culture nodes). Theoretically, the subsequent setting steps in the first grouping method and the second grouping method are the same, and the only difference between the two is that because the culture water monitored and controlled through the first grouping method is correlated (belonging to the same culture pond), the terminal host 720 controls the smart culture nodes in the same culture pond through a special mechanism. Regarding the second grouping method, the terminal host 720 simply issues a control instruction to set all the smart culture nodes.

Then, in step S803 (or S813), the culturist selects the identifiers of the smart culture nodes which are to be grouped into the same culture group. Next, in step S805 (or S815), the culturist selects the water quality information to be detected (for example, water temperature and dissolved oxygen content). Thereafter, in step S807 (or S817), the culturist inputs the expected environmental parameters. After that, in step S809 (or S819), the culture gateway 730 sends an identifier of the culture group according to a control instruction of the terminal host 720, and in step S811 (or S821), the culture gateway 730 sends the water quality information to be detected.

Referring to FIG. 7 again, in the present embodiment, the smart culture nodes 701˜706 are connected to the culture gateway 730 through a control network 752 (e.g. a control area network), and the terminal host 720 is connected to the culture gateway 730 through a communication network 754. Thus, in the present embodiment, the terminal host 720 remotely controls the smart culture nodes 701˜706 connected through the control network 752 through the relay of the communication network and the culture gateway 730. In an embodiment of the present invention, the communication network 754 is an IP communication network; however, a non-IP communication network may also be applied to the present invention.

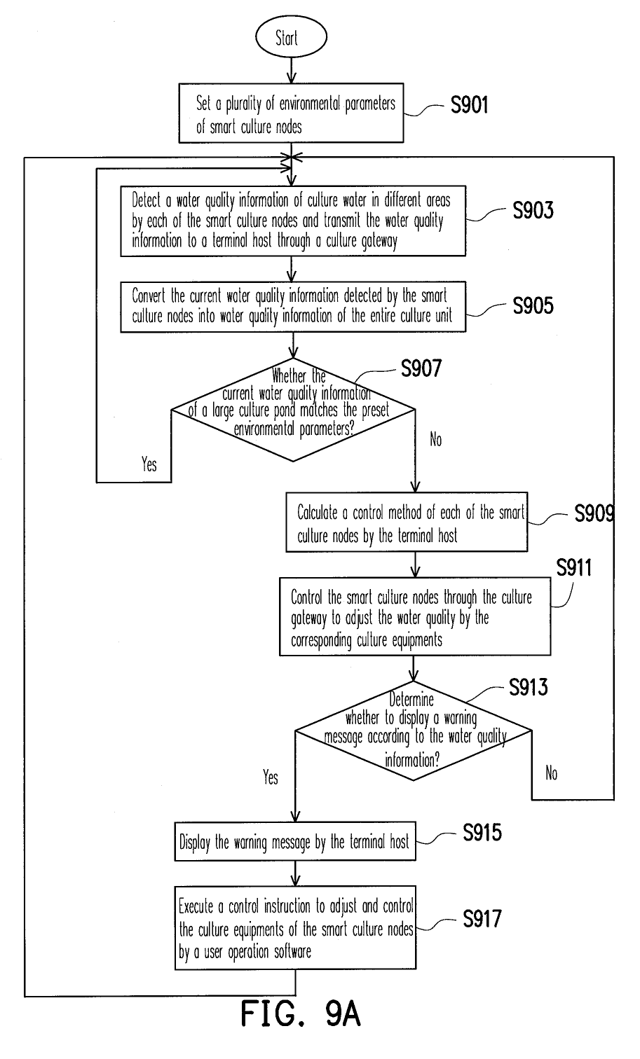

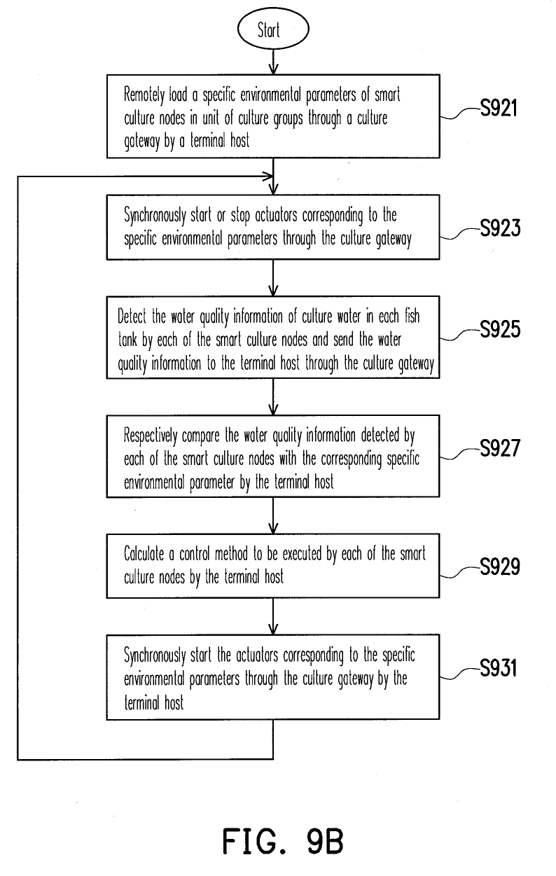

FIG. 9A illustrates a method for monitoring and controlling the quality of a culture water, wherein the smart culture nodes are grouped through the first grouping method.

Referring to FIG. 9A, in step S901, the terminal host 720 remotely sets a plurality of environmental parameters of the smart culture nodes 701˜703 in unit of culture groups through the culture gateway 730. Then, in step S903, the water quality information of the culture water in each area is detected by using each of the smart culture nodes 701˜703 and is sent to the terminal host 720 through the culture gateway 730.

Next, in step S905, the terminal host 720 converts the current water quality information detected by the smart culture nodes 701˜703 into a water quality information related to the entire culture unit (i.e., the large culture pond). After that, in step S907, the terminal host 720 compares the current water quality information of the large culture pond with the preset environmental parameters. If the terminal host 720 determines in step S907 that the current water quality information does not match the preset environmental parameters, then in step S909, the terminal host 720 calculates a control method for each of the smart culture nodes 701˜703, and in step S911, the terminal host 720 controls the smart culture nodes 701˜703 through the culture gateway 730 so as to adjust the water quality by using the corresponding culture equipments. If it is determined in step S907 that the current water quality information matches the preset environmental parameters, the terminal host 720 takes no action and step S903 is executed again after a certain time to continue with the monitoring and controlling task.

In addition, in step S901, the culturist may further set a prompt setting of the water quality information besides setting the environmental parameters. For example, when the temperature exceeds an expected value, the turbidity exceeds an expected value, the concentration of a compound exceeds an expected value, or the content of a specific element is too low, etc, the smart culture nodes 701˜703 sends the information to the terminal host 720 to prompt the culturist. Thus, in step S913, the terminal host 720 determines whether to display a warning message to the culturist according to the water quality information.

If it is determined in step S913 that the culturist is not to be prompted, step S903 is executed. If it is determined in step S913 that the culturist is to be prompted, in step S915, the terminal host 720 displays the warning message, and in step S917, the culturist executes a control instruction through the user operation software to adjust and control various culture equipments of the smart culture nodes 701˜703, and step S903 is then executed.

Similar to that in the first embodiment, in another embodiment of the present invention, the water quality monitoring and controlling system 700 may further include a environment learning module for recording the regulation process carried out in step S917 so that when subsequently a similar situation is encountered, the terminal host 720 can set the smart culture nodes 701˜703 according to the recorded regulation process.

FIG. 9B illustrates a method for monitoring and controlling the quality of a culture water, wherein the smart culture nodes are grouped through the second grouping method.

Referring to FIG. 9B, in step S921, the terminal host 720 remotely loads a specific environmental parameter (for example, the water temperature) of the smart culture nodes 704˜706 in unit of culture groups through the culture gateway 730. In step S923, the actuators corresponding to the specific environmental parameter are synchronously started or stopped through the culture gateway 730.

After that, in step S925, the water quality information of the culture water in each of the fish tanks is respectively detected by using each of the smart culture nodes 704˜706 and is sent to the terminal host 720 through the culture gateway 730. In step S927, the terminal host 720 respectively compares the water quality information detected by each of the smart culture nodes 704˜706 with the corresponding specific environmental parameter.

Thereafter, in step S929, the terminal host 720 calculates a control method for each of the smart culture nodes 704˜706, and in step S931, the terminal host 720 synchronously starts the actuators corresponding to the specific environmental parameter through the culture gateway 730, and then step S923 is executed to continue with the monitoring and controlling task.

As described above, in a water quality monitoring and controlling system provided by the present invention, smart culture nodes having integrated water quality analyzers and a culture gateway are adopted such that water quality information to be detected is remotely set in unit of groups. In addition, in a water quality monitoring and controlling system provided by the present invention, an automatic optical analyzer is adopted such that the quality of a culture water can be automatically analyzed by using water quality reagents and optical devices. In particular, regarding a large culture unit, by monitoring and controlling the smart culture nodes in unit of groups, both the time and manpower can be greatly reduced and a scientific and real-time management can be achieved.

Moreover, in a water quality monitoring and controlling system provided by the present invention, an environment learning module is further adopted such that the regulation process made by a culturist can be recorded and subsequently used in similar culture environment. For example, a culturist discovers that the pH value of a culture water in a float glass tank is 7.2 after the water is just changed. Even though at this time the smart culture node automatically provides carbon dioxide, if the culturist does not want the concentration of carbon dioxide to be too high and accordingly affect the float grass but at the same time hope the pH value to drop within an expected range, the culturist can manually issue an instruction through a terminal host to start a water softener. With the operation of the water softener and the provision of carbon dioxide, the pH value can quickly increase to an ideal value. The environment learning module records foregoing manual operations as a record file so that the record file can be directly used when later one a similar situation is encounters or updated into one method for controlling pH value. By adopting the environment learning module, the efficiency in system management is improved and reference can be provided to other culturists through the recording of the regulation process.

Furthermore, the water quality monitoring and controlling system provided by the present invention can be applied regardless of whether a single culture node or multiple culture nodes are adopted. For example, when a culturist breeds a kind of fish in an experimental tank in order to improve the productivity or body color thereof and the culture condition in the experimental tank reaches an ideal state, a regulation process of a smart culture node in the experimental tank is shared among other experimental tanks through a culture gateway. In addition, the regulation process can be exchanged with other culturists as culture experiences or served as a value added service of the water quality monitoring and controlling system. Thereby, consumers who do not know anything about aquaculture or have no time for managing a culture environment can still carry out culture tasks by using the setting files provided by a manufacturer. Besides, the water quality monitoring and controlling system described above also allows the manufacturer to directly manage a fish tank installed in a consumer's house through an IP network.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A system for monitoring and controlling the quality of a culture water, comprising:
a plurality of smart culture nodes, wherein each of the smart culture nodes is connected to a culture equipment and comprises:
a water quality parameter regulator, for setting an environmental parameter;
an integrated water quality analyzer, electrically connected to the water quality parameter regulator, for detecting a water quality information of the culture water; and
an actuator, electrically connected to the water quality parameter regulator, for starting the culture equipment;
a terminal host, for issuing a control instruction to set the environmental parameters of the smart culture nodes; and
a culture gateway, connected between the smart culture nodes and the terminal host for relaying the water quality information and the control instruction,
wherein the terminal host controls the culture gateway to group at least a part of the smart culture nodes into a culture group and synchronously controls the smart culture nodes in the culture group to start the culture equipments according to the water quality information collected by the smart culture nodes in the culture group.

2. The system according to claim 1, further comprising an environment learning module, wherein the environment learning module records a regulation process for setting the environmental parameters.

3. The system according to claim 1, further comprising:
a communication network, for connecting the culture gateway and the terminal host; and
a control network, for connecting the smart culture nodes and the culture gateway.

4. The system according to claim 3, wherein the culture gateway comprises:
a data relay unit, for relaying data between the communication network and the control network; and
a group decision-making unit, for grouping the smart culture nodes in the culture group according to the control instruction received from the terminal host to synchronously control the smart culture nodes in the culture group.

5. The system according to claim 1, wherein the integrated water quality analyzer comprises at least one sensor for detecting the water quality information of the culture water.

6. The system according to claim 1, wherein the integrated water quality analyzer comprises an automatic optical water quality analyzer, and the automatic optical water quality analyzer comprises:
a water quality analysis controller;
a water extraction structure, electrically connected to the water quality analysis controller, for extracting the culture water through a water extraction pipeline;
a test water guiding structure, electrically connected to the water quality analysis controller, for obtaining the culture water from the water extraction structure through a water pipeline and injecting the culture water into at least one test container;
a reagent guiding structure, electrically connected to the water quality analysis controller and having at least one water quality reagent, wherein the reagent guiding structure injects the at least one water quality reagent into the at least one test container; and
an optical analysis device, electrically connected to the water quality analysis controller, for analyzing the culture water in the test container to obtain the water quality information.

7. The system according to claim 1, wherein each of the water quality parameter regulators comprises:
a parameter decision-making unit, for sorting the water quality information; and
a water quality management unit, coupled to the parameter decision-making unit, for controlling the corresponding actuators.

8. The system according to claim 2, wherein the environment learning module comprises:
a parameter recording unit, for recording the regulation process; and
an automatic adjusting unit, coupled to the parameter recording unit, for setting the environmental parameters of the smart culture nodes according to the regulation process.

9. The system according to claim 1, wherein the environmental parameters selected from the group consisting of a culture unit water volume, a dissolved oxygen content, a carbon dioxide concentration, a pH value, an illumination pattern, a feed quantity, and a temperature range.

10. The system according to claim 1, wherein the terminal host comprises a prompt unit for displaying a concentration prompt, a compound prompt, or an element content prompt according to the water quality information.

11. The system according to claim 1, wherein the culture equipments selected from the group consisting of at least one of a lamp, an electromagnetic valve for controlling carbon dioxide, a heating bar, an automatic feeder, a air pump, or a water cooler.

12. The system according to claim 6, wherein the water quality reagent comprises a nitrogen compound reagent or a dissolved oxygen reagent.

13. The system according to claim 5, wherein the at least one sensor comprises at least one of a pH sensor and a temperature sensor.

14. A method for monitoring and controlling the quality of a culture water, suitable for a system for monitoring and controlling the quality of the culture water, wherein the system comprises a terminal host, a plurality of smart culture nodes, and a culture gateway, and each of the smart culture nodes is connected to a culture equipment, the method comprising:
remotely setting a plurality of environmental parameters of the smart culture nodes through the culture gateway by using the terminal host;
detecting a water quality information by using each of the smart culture nodes; and
controlling the culture gateway to group at least a part of the smart culture nodes into a culture group and synchronously controlling the smart culture nodes in the culture group according to the water quality information collected by the smart culture nodes in the culture group to start the culture equipments by using the terminal host.

15. The method according to claim 14, further comprising recording a regulation process for setting the environmental parameters by using an environment learning module of the system.

16. A system for monitoring and controlling the quality of a culture water, comprising:
a plurality of actuators, respectively connected to a plurality of culture equipments;
a water quality parameter regulator, electrically connected to the actuators, and having a plurality of environmental parameters corresponding to the actuators;
an integrated water quality analyzer, electrically connected to the water quality parameter regulator, for detecting a water quality information of the culture water;
a terminal host, for issuing a control instruction to set the environmental parameters; and
a culture gateway, connected between the water quality parameter regulator and the terminal host, for relaying the control instruction,
wherein the water quality parameter regulator activates the actuators to start the culture equipments according to the water quality information and the environmental parameters.

17. The system according to claim 16, wherein the integrated water quality analyzer comprises at least one sensor electrically connected to the water quality parameter regulator for detecting the water quality information of the culture water.

18. The system according to claim 16, wherein the integrated water quality analyzer comprises an automatic optical water quality analyzer electrically connected to the water quality parameter regulator, and the automatic optical water quality analyzer comprises:
a water quality analysis controller;
a water extraction structure, electrically connected to the water quality analysis controller, for extracting the culture water through a water extraction pipeline;
a test water guiding structure, electrically connected to the water quality analysis controller, for obtaining the culture water from the water extraction structure through a water pipeline and injecting the culture water into at least one test container;
a reagent guiding structure, electrically connected to the water quality analysis controller and having at least one water quality reagent, wherein the reagent guiding structure injects the at least one water quality reagent into the at least one test container; and
an optical analysis device, electrically connected to the water quality analysis controller, for analyzing the culture water in the test container so as to obtain the water quality information.

19. The system according to claim 18, wherein the environmental parameters selected from the group consisting of at least one of a culture unit water volume, a dissolved oxygen content, a carbon dioxide concentration, a pH value, an illumination pattern, a feed quantity, a temperature range, and an actuator active time.

20. The system according to claim 18, wherein the culture equipments selected from the group consisting of at least one of a lamp, an electromagnetic valve for controlling carbon dioxide, a heating bar, an automatic feeder, a air pump, and a water cooler.

21. The system according to claim 18, wherein the at least one water quality reagent comprises at least one of a nitrogen compound reagent or a dissolved oxygen reagent.

22. The system according to claim 18, wherein the at least one sensor comprises at least one of a pH sensor or a temperature sensor.

23. A method for monitoring and controlling the quality of a culture water, suitable for the system in claim 16, the method comprising:
setting the environmental parameters of the water quality parameter regulator;
obtaining the water quality information through the integrated water quality analyzer;
comparing the water quality information with the environmental parameters to obtain a comparison result; and
determining whether to activate the actuators to start the culture equipments according to the comparison result.

24. An integrated water quality analyzer, comprising:
a water quality analysis controller;
a water extraction structure, electrically connected to the water quality analysis controller for extracting a test water through a water extraction pipeline;
a test water guiding structure, electrically connected to the water quality analysis controller, for obtaining the test water from the water extraction structure through a water pipeline and injecting the test water into at least one test container;
a reagent guiding structure, electrically connected to the water quality analysis controller and having at least one water quality reagent, wherein the reagent guiding structure injects the at least one water quality reagent into the at least one test container; and
an optical analysis device, electrically connected to the water quality analysis controller, for analyzing the test water in the test container to obtain a water quality information.

25. The integrated water quality analyzer according to claim 24, further comprising at least one sensor for detecting the test water.

26. The integrated water quality analyzer according to claim 24, further comprising a turntable for placing the at least one test container.

27. The integrated water quality analyzer according to claim 24, wherein the at least one water quality reagent comprises at least one of a nitrogen compound reagent or a dissolved oxygen reagent.

28. The integrated water quality analyzer according to claim 24, wherein the at least one sensor comprises at least one of a pH sensor or a temperature sensor.

29. A water quality analysis method, suitable for the integrated water quality analyzer in claim 24, the method comprising:
controlling the water extraction structure to obtain a test water through the water extraction pipeline by using the water quality analysis controller;
injecting the test water into the at least one test container by using the water extraction structure;
automatically injecting the at least one water quality reagent into the corresponding test container by using the reagent guiding structure; and
analyzing the test water in the test container by using the optical analysis device to generate a water quality information of the test water.
